(19) United States
(12) Patent Application Publication
(10) Pub. No.: US 2016/0128931 A1
(43) Pub. Date: May 12, 2016

(54) Q. Hair Regrowth Spray (Q. Hair Spray)

(71) Applicant: Levy Quinn Barnes, JR., Port Arthur, TX (US)

(72) Inventor: Levy Quinn Barnes, JR., Port Arthur, TX (US)

(21) Appl. No.: 14/539,350

(22) Filed: Nov. 12, 2014

(51) Int. Cl. A61K 8/97; A61K 8/92; A61Q 7/00; A61K 8/365

(52) U.S. Cl. CPC A61K 8/97; A61K 8/365; A61K 8/922; A61Q 7/00; A61K 2800/5922

(57) ABSTRACT

Q. Hair Regrowth Spray (Q. Hair Spray) is a combination of Dried Lavender, Dried Rosemary, Chopped Ginger, Dried Sage, Evaporated Cane Juice, Soybean oil, Extra Virgin Olive Oil, and Citric Acid boiled in distilled water. The combination of these ingredients suppresses Dihydrotestoserone , reopens the hair follicles and allows the papilla to grow hair again. This treats multiple forms of Alopecia. Q. Hair Regrowth Spray (Q. Hair Spray) allows the hair to grow back from bald and thin areas on the scalp. This was created by Dr. Levy Q. Barnes, Jr

This is a natural topical solution for hair to stop Dihydrotestoserone from causing baldness in males and females and regrows hair. For hair follicles that have been damaged due to chemical burns, traction from harsh hair styles and general damage, Q. Hair Regrowth Spray and its ingredients rejuvenate the components in the follicle and allows an opportunity for the hair to grow back. Lastly it helps the hair grow hair quicker and supports long hair length.

This is a hair solution that slows and stops the formation of Dihydrotestoserone, which is the main factor in male and female pattern baldness. It also reopens and revitalizes the hair follicle, nourishes the bulb, and allows the papilla to produce and to grow hair in men and women.

Ingredients: Distilled water, Boiled Dried Lavender, Boiled Dried Rosemary, Boiled Minced Ginger, Evaporated Cane Juice, Soybean oil, Extra Virgin Olive Oil, Dried Sage, and Citric Acid.

### REFERENCES

“What is DHT? What is DHT's Role in Baldness” www.medicalnewstoday.com Thursday Feb. 23, 2012
“Lavender Overview Information” & “Ginger Overview Information” Natural Medicines Comprehensive Database Consumer Version© Therapeutic Research Faculty 2009.
“How About Rosemary” Jun. 16, 2009 www.chemicaloftheday.squarespace.com Copyright & copy; 2009, [Stephanie Greenwood].

### FIELD OF INVENTION

This invention uses natural remedies to suppress DHT in the scalp in the form of a topical spray and cause healthy hair growth.

### DESCRIPTION OF RELATED ARTICLE

The are multiple reasons for hair loss. The main reason for hair loss is caused by DHT which is the main factor in male pattern baldness. This hormone affects males and females by causing hair loss, by causing the hair follicles to shrink in the frontal and vertex portions of the scalp. Males are more susceptible to DHT at an early age because their bodies do not contain as much estrogen as females do. Estrogen naturally fights against DHT. When females begin to go through menopause or if there are other factors which cause their estrogen levels to drop, their scalps can begin to form in their scalps which causes their hair follicles to shrink and lose their hair.

The compounds in the 20-40% of lavender, 20-40% of rosemary, 15-20% of ginger,5-10% of dried raw sage, and 1-5% of Evaporated Cane juice, Soybean oil, Extra Virgin Olive Oil and citric acid boiled in distilled water in each solution causes DHT to be suppressed and allows the hair follicle to rejuvenate and thrive by causing hair to grow. When applying these hormones to a watery solution it can saturate the scalp and allow for easy absorption. Other patents who have utilized these ingredients have used only oils. Oils do not mix in easily through our pores nor are they able to absorb into our follicles and fully alter the effects of DHT as easily as they can by water. By distributing the solution in water we are able to allow the solution to absorb into the scalp and cause promotion for hair growth.

### SUMMARY OF PRODUCT

This solution, which contains 20-40% Dried Lavender, 20-40% Dried Rosemary, 15-20% Ginger, 5-10% of Raw dried Sage, and 1-5% of Evaporated Cane juice, Soybean oil, Extra Virgin Olive oil, and Citric Acid in at least 100 degrees of distilled water. This combination naturally suppresses DHT in the scalp and opens up the follicles. By applying it topically in the form of a spray this solution allows the bulbs in the follicle to regenerate and also allows papilla to produce hair to grow. It also causes hair to grow quicker allowing it to stay in the Anagen phase longer.

By suppressing DHT this solution ceases hair loss and allows the components of the hair follicle to rejuvenate and regenerate a healthy hair. For hair follicles that have shrank, our solution cancels the effects that DHT has caused to the hair follicle and helps to reopen the follicles. After this it reverses the damage and causes the hair follicle to almost completely resurrect.

There are no current patents that use this combination nor percentage of the solution we have mixed with water.

In order to make this product we place 1 cup of dried Lavender, 1 cup of Dried Rosemary, half a cup of minced Ginger mixed with Evaporated Cane Juice, Soybean Oil, and Extra Virgin Olive oil, and 2 tablespoons of raw Dried Sage in 2 gallons of distilled water. Boil the distilled water for 20 minutes until the ingredients appear soggy. Allow to cool for one hour. Then strain out the liquid and bottle in a spray bottle. The solution then needs to be refrigerated. This solution cannot remain at room temperature for over a month. After bottle is refrigerated it will last for up to 3 months. After then solution is no good and must be discarded.

The user should apply 1-3 times daily by spraying the solution to the scalp in the affected areas until it is moist with the solution. To attain maximum growth the user may use product 3 times a day. After a week of maximum use, most users see results in thinning and bald areas of the vertex and frontal portions of scalp.

Q. Hair Regrowth Spray (Q. Hair Spray) invented by Dr. Levy Q. Barnes, Jr.

1. I claim that Q. Hair Regrowth Spray is made up of dried lavender, dried rosemary, dried sage, evaporated cane juice, citric acid, sage, and extra virgin olive oil.

2. I claim that Q. Hair Regrowth Spray is a natural topical spray that treats multiple forms of Alopecia.

3. I claim that Q. Hair Regrowth Spray is a natural topical spray that re-grows hair from bald and thin areas.

4. I claim that Q. Hair Regrowth Spray grows hair quicker.

5. I claim that Q. Hair Regrowth Spray supports growth of long and full hair.

6. I claim that Q. Hair Regrowth Spray detoxifies the scalp.

7. I claim that Q. Hair Regrowth Spray assists in the repair of split ends.
